(19) United States
(12) Patent Application Publication
(10) Pub. No.: US 2021/0043328 A1
(43) Pub. Date: Feb. 11, 2021

(54) SYSTEM AND METHOD FOR PROVIDING MODEL-BASED POPULATION INSIGHT GENERATION

(71) Applicant: KONINKLIJKE PHILIPS N.V., EINDHOVEN (NL)

(72) Inventors: Jan Johannes Gerardus DE VRIES, LEENDE (NL); Joep Joseph Benjamin Nathan VAN BERKEL, LIMBURG (NL); Ioanna SOKORELI, EINDHOVEN (NL)

(21) Appl. No.: 16/969,122

(22) PCT Filed: Feb. 12, 2019
(86) PCT No.: PCT/EP2019/053368

§ 371 Date: Aug. 11, 2020

Related U.S. Application Data
(60) Provisional application No. 62/632,141, filed on Feb. 19, 2018.

(51) Int. Cl. G16H 50/70; G16H 40/20; G16H 50/20; G16H 10/60; G16H 10/20

(52) U.S. Cl. CPC G16H 50/70; G16H 40/20; G16H 10/20; G16H 10/60; G16H 50/20

(57) ABSTRACT

The present disclosure pertains to a system for providing model-based population insight generation. In some embodiments, the system (i) obtains a data collection representative of a population of individuals; (ii) determines a grouping of the data collection to obtain groups representative of a plurality of individuals each having at least one attribute of a plurality of attributes; (iii) determines (a) a statistic for each attribute of the plurality of attributes for each of the groups, (b) whether there is a difference between the statistic of an attribute of a group and the statistics of the attribute of the other groups, and (c) whether a measure of significance for each difference of the differences exceeds a predetermined threshold; (iv) generate insight information reflecting the difference between a type of individual and other types of individuals relative to the attribute; and (v) effectuates presentation of the insight information.

### BACKGROUND

### 1. Field

The present disclosure pertains to a system and method for providing population insight generation.

### 2. Description of the Related Art

Population health analytics solutions aim at collecting available data on a certain population for which a care provider is accountable and analyzing the data for identifying groups of patients to gain insight into the issues related to providing care (e.g., outcomes, engagement with healthcare system, access to care, costs, etc.). Although computer-assisted insight generation systems exist, such systems may not facilitate generation of accurate insight information due to such systems not utilizing data on other important determinants of health (e.g., beside clinical information). For example, prior art systems may facilitate (i) filtering of and (ii) rule-based searching on population data, thus requiring care managers to perform manual inspection of results of the filtered data and rule-based searches for the selected segmented population data by relying on their understanding and knowledge of the related care problems and current means of care provision. These and other drawbacks exist.

### SUMMARY

Accordingly, one or more aspects of the present disclosure relate to a system for providing model-based population insight generation. The system comprises one or more processors configured by machine readable instructions and/or other components. The one or more hardware processors are configured to: obtain a data collection representative of a population of individuals; determine a grouping of the data collection to obtain groups representative of a plurality of individuals, each of the plurality of individuals having at least one attribute of a plurality of attributes; determine a statistic for each attribute of the plurality of attributes for each of the groups; for each attribute of the plurality of attributes, determine whether there is a difference between the statistic of an attribute of a group and the statistics of the attribute of the other groups; determine whether a measure of significance for each difference of the differences exceeds a predetermined threshold; generate, for each difference of the differences that has the measure of significance exceeding the predetermined threshold, insight information, the insight information reflecting the difference between a type of individual and other types of individuals relative to the attribute, the type of individual being associated with the group, and the other types of individuals being associated with at least one other group; and effectuate, via a user interface, presentation of the insight information.

Another aspect of the present disclosure relates to a method for providing model-based population insight generation with a generation system. The system comprises one or more processors configured by machine readable instructions and/or other components. The method comprises: obtaining, with one or more processors, a data collection representative of a population of individuals; determining, with the one or more processors, a grouping of the data collection to obtain groups representative of a plurality of individuals, each of the plurality of individuals having at least one attribute of a plurality of attributes; determining, with the one or more processors, a statistic for each attribute of the plurality of attributes for each of the groups; for each attribute of the plurality of attributes, determining, with the one or more processors, whether there is a difference between the statistic of an attribute of a group and the statistics of the attribute of the other groups; determining, with the one or more processors, whether a measure of significance for each difference of the differences exceeds a predetermined threshold; for each difference of the differences that has the measure of significance exceeding the predetermined threshold, generating, with the one or more processors, insight information, the insight information reflecting the difference between a type of individual and other types of individuals relative to the attribute, the type of individual being associated with the group, and the other types of individuals being associated with at least one other group; and effectuating, via a user interface, presentation of the insight information.

Still another aspect of present disclosure relates to a system for providing model-based population insight generation. The system comprises means for obtaining a data collection representative of a population of individuals; means for determining a grouping of the data collection to obtain groups representative of a plurality of individuals, each of the plurality of individuals having at least one attribute of a plurality of attributes; means for determining a statistic for each attribute of the plurality of attributes for each of the groups; for each attribute of the plurality of attributes, means for determining whether there is a difference between the statistic of an attribute of a group and the statistics of the attribute of the other groups; means for determining whether a measure of significance for each difference of the differences exceeds a predetermined threshold; for each difference of the differences that has the measure of significance exceeding the predetermined threshold, means for generating insight information, the insight information reflecting the difference between a type of individual and other types of individuals relative to the attribute, the type of individual being associated with the group, and the other types of individuals being associated with at least one other group; and means for effectuating presentation of the insight information.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system 10 configured for providing population insight generation.

FIG. 2 illustrates various options for patient/population exploration, in accordance with one or more embodiments.

FIG. 3 illustrates hierarchical clustering of a population, in accordance with one or more embodiments.

FIG. 4 illustrates different groups of patients having similar clinical conditions, in accordance with one or more embodiments.

FIG. 5 illustrates insight information generated for a cluster vs. a complement, in accordance with one or more embodiments.

FIG. 6 illustrates insight information generated for a cluster vs. a complement that specifies the particular attribute of comparison, in accordance with one or more embodiments.

FIG. 7 illustrates insight information generated for a cluster vs. other peer clusters, in accordance with one or more embodiments.

FIG. 8 illustrates a method for providing model-based population insight generation, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

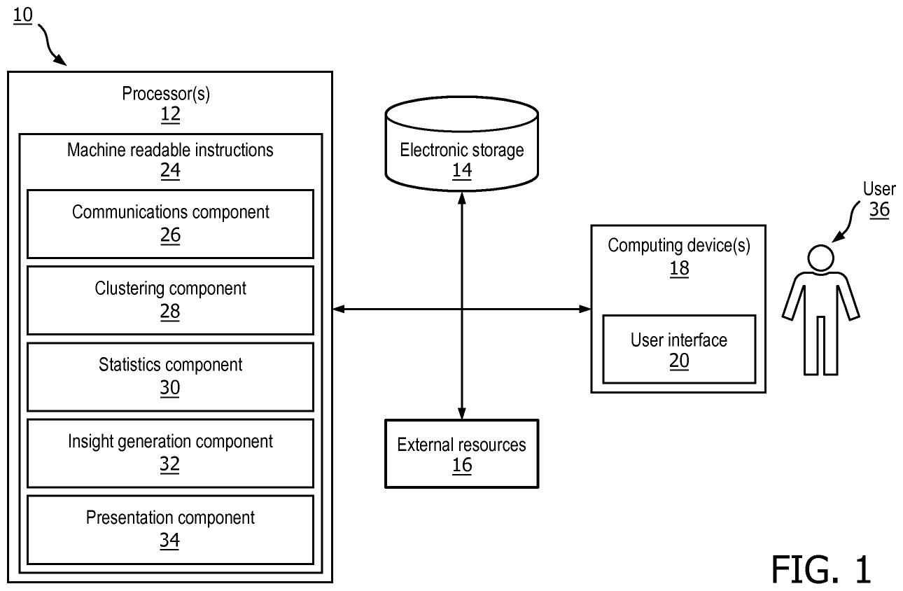

FIG. 1 is a schematic illustration of a system 10 configured for providing population insight generation. In some embodiments, system 10 is configured to (i) identify one or more groups of individuals having one or more attributes (clustering), (ii) determine a statistic for each one of the one or more attributes (e.g., mean, median, mode, variance, standard deviation, trend), (iii) compare the statistics to determine a difference having a measure of significance exceeding a predetermined threshold, and (iv) select the most (clinically) relevant differences to generate insight information. In some embodiments, system 10 facilitates care optimization through automatic generation or extraction of clinically meaningful and actionable insight information from a growing amount of data in order to be able to identify care needs of (sub) populations and act upon these insights, thereby improving health outcomes and reduce costs.

In some embodiments, system 10 is configured to (i) perform clustering on a population data collection to obtain groups representative of a plurality of individuals and (ii) generate insight information applicable to the groups. In some embodiments, system 10 is configured to perform the clustering, the insight information generation, or other operations described herein via one or more prediction models. Such prediction models may include neural networks, other machine learning models, or other prediction models. As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

In some embodiments, system 10 comprises processors 12, electronic storage 14, external resources 16, computing device 18 (e.g., associated with user 36), or other components.

Electronic storage 14 comprises electronic storage media that electronically stores information (e.g., data collection representative of a population of individuals). The electronic storage media of electronic storage 14 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 14 may be (in whole or in part) a separate component within system 10, or electronic storage 14 may be provided (in whole or in part) integrally with one or more other components of system 10 (e.g., computing device 18, etc.). In some embodiments, electronic storage 14 may be located in a server together with processors 12, in a server that is part of external resources 16, and/or in other locations. Electronic storage 14 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 14 may store software algorithms, information determined by processors 12, information received via processors 12 and/or graphical user interface 20 and/or other external computing systems, information received from external resources 16, and/or other information that enables system 10 to function as described herein.

External resources 16 include sources of information and/or other resources. For example, external resources 16 may include a population's electronic medical record (EMR), the population's electronic health record (EHR), or other information. In some embodiments, external resources 16 include health information related to the population. In some embodiments, the health information comprises demographic information, vital signs information, medical condition information indicating medical conditions experienced by individuals in the population, treatment information indicating treatments received by the individuals, and/or other health information. In some embodiments, external resources 16 include sources of information such as databases, websites, etc., external entities participating with system 10 (e.g., a medical records system of a health care provider that stores medical history information of patients), one or more servers outside of system 10, and/or other sources of information. In some embodiments, external resources 16 include components that facilitate communication of information such as a network (e.g., the internet), electronic storage, equipment related to Wi-Fi technology, equipment related to Bluetooth® technology, data entry devices, sensors, scanners, and/or other resources. In some embodiments, some or all of the functionality attributed herein to external resources 16 may be provided by resources included in system 10.

Processors 12, electronic storage 14, external resources 16, computing device 18, and/or other components of system 10 may be configured to communicate with one another, via wired and/or wireless connections, via a network (e.g., a local area network and/or the internet), via cellular technology, via Wi-Fi technology, and/or via other resources. It will be appreciated that this is not intended to be limiting, and that the scope of this disclosure includes embodiments in which these components may be operatively linked via some other communication media. In some embodiments, processors 12, electronic storage 14, external resources 16, computing device 18, and/or other components of system 10 may be configured to communicate with one another according to a client/server architecture, a peer-to-peer architecture, and/or other architectures.

Computing device 18 may be configured to provide an interface between user 36 and/or other users, and system 10. In some embodiments, computing device 18 is and/or is included in desktop computers, laptop computers, tablet computers, smartphones, smart wearable devices including augmented reality devices (e.g., Google Glass), wrist-worn devices (e.g., Apple Watch), and/or other computing devices associated with user 36, and/or other users. In some embodiments, computing device 18 facilitates presentation of possible insight information, insight information, or other information. In some embodiments, computing device 18 facilitates obtaining user input related to the user 36's preference of the possible insight information. Accordingly, computing device 18 comprises a user interface 20. Examples of interface devices suitable for inclusion in user interface 20 include a touch screen, a keypad, touch sensitive or physical buttons, switches, a keyboard, knobs, levers, a camera, a display, speakers, a microphone, an indicator light, an audible alarm, a printer, tactile haptic feedback device, or other interface devices. The present disclosure also contemplates that computing device 18 includes a removable storage interface. In this example, information may be loaded into computing device 18 from removable storage (e.g., a smart card, a flash drive, a removable disk, etc.) that enables caregivers or other users to customize the implementation of computing device 18. Other exemplary input devices and techniques adapted for use with computing device 18 or the user interface include an RS-232 port, RF link, an IR link, a modem (telephone, cable, etc.), or other devices or techniques.

Processor 12 is configured to provide information processing capabilities in system 10. As such, processor 12 may comprise one or more of a digital processor, an analog processor, a digital circuit designed to process information, an analog circuit designed to process information, a state machine, or other mechanisms for electronically processing information. Although processor 12 is shown in FIG. 1 as a single entity, this is for illustrative purposes only. In some embodiments, processor 12 may comprise a plurality of processing units. These processing units may be physically located within the same device (e.g., a server), or processor 12 may represent processing functionality of a plurality of devices operating in coordination (e.g., one or more servers, computing device, devices that are part of external resources 16, electronic storage 14, or other devices.)

As shown in FIG. 1, processor 12 is configured via machine-readable instructions 24 to execute one or more computer program components. The computer program components may comprise one or more of a communications component 26, a clustering component 28, a statistics component 30, an insight generation component 32, a presentation component 34, or other components. Processor 12 may be configured to execute components 26, 28, 30, 32, or 34 by software; hardware; firmware; some combination of software, hardware, or firmware; or other mechanisms for configuring processing capabilities on processor 12.

It should be appreciated that although components 26, 28, 30, 32, and 34 are illustrated in FIG. 1 as being co-located within a single processing unit, in embodiments in which processor 12 comprises multiple processing units, one or more of components 26, 28, 30, 32, or 34 may be located remotely from the other components. The description of the functionality provided by the different components 26, 28, 30, 32, or 34 described below is for illustrative purposes, and is not intended to be limiting, as any of components 26, 28, 30, 32, or 34 may provide more or less functionality than is described. For example, one or more of components 26, 28, 30, 32, or 34 may be eliminated, and some or all of its functionality may be provided by other components 26, 28, 30, 32, or 34. As another example, processor 12 may be configured to execute one or more additional components that may perform some or all of the functionality attributed below to one of components 26, 28, 30, 32, or 34.

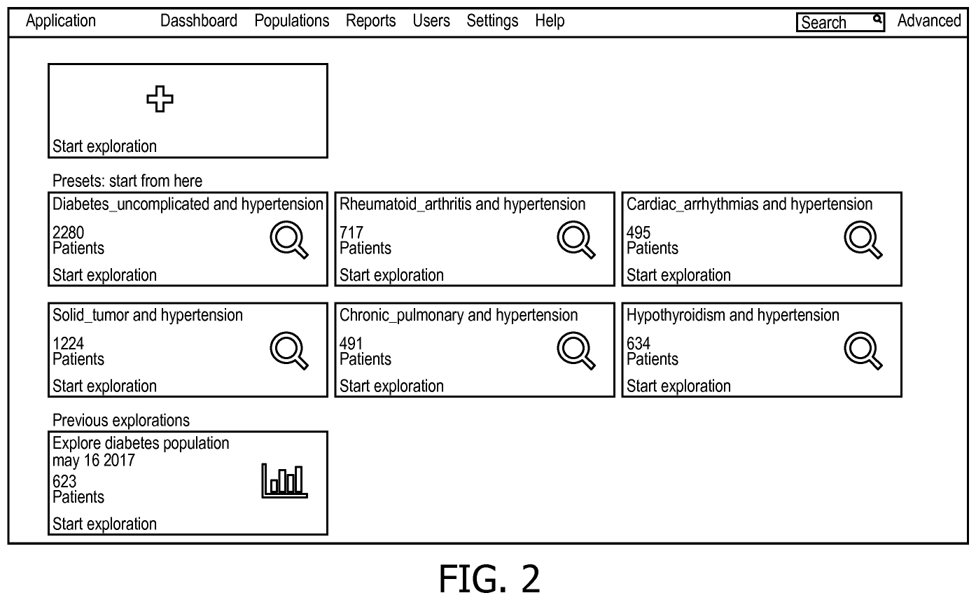

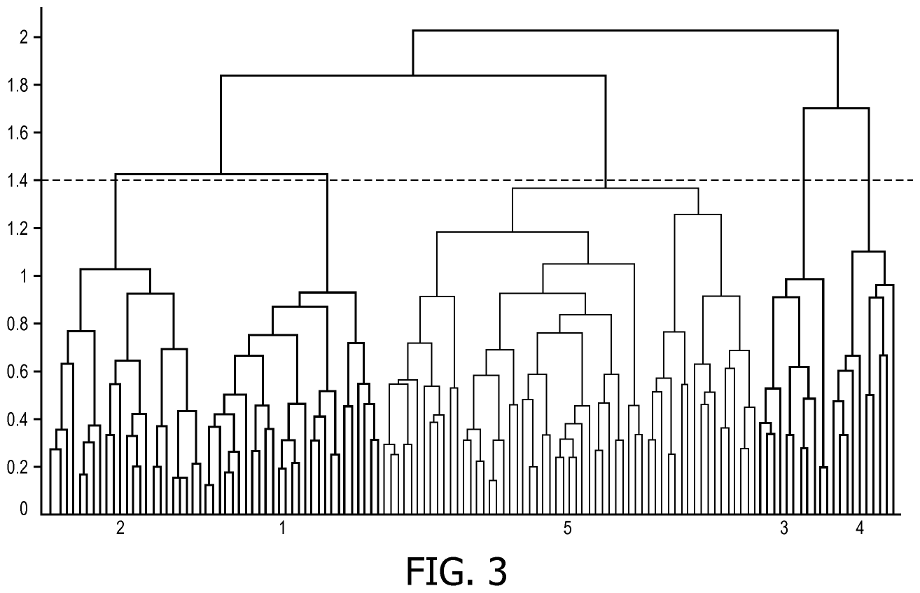

Communications component 26 is configured to obtain a data collection representative of a population of individuals. In some embodiments, the data collection may be representative of 100 or more individuals, 1,000 or more individuals, 10,000 or more individuals, 100,000 or more individuals, 1,000,000 or more individuals, 100,000,000 or more individuals, or other number of individuals. In some embodiments, the data collection may include health information corresponding to the individuals. In some embodiments, the health information indicates (i) physiological conditions of the individuals, (ii) treatments provided to the individuals respectively for the physiological conditions, (iii) whether such treatments were successful in treating the individuals, (iv) the levels of such success in treating the individuals, or (v) other information. In some embodiments, the data collection is obtained based on the stored data collection in electronic storage 14. In some embodiments, the data collection is obtained via external resources 16. In some embodiments, the data collection is obtained via a query to external resources 16 based on one or more criteria. In some embodiments, the query is based on one or more physiological, demographic, or other parameters of an individual. In one embodiment, the present disclosure comprises means for obtaining a data collection representative of a population of individuals, with such means for obtaining the data collection taking the form of communications component 26. By way of a non-limiting example, FIG. 2 illustrates various options for patient/population exploration, in accordance with one or more embodiments. As shown in FIG. 2, user 36 may start the patient/population exploration from a blank exploration starting point. Furthermore, system 10 may facilitate user 36 to start from an automatically generated start scenario for exploration. For example, communications component 26 may form one or more queries based on the most recurring medical conditions in a healthcare facility. In this example, a data collection representative of a plurality of individuals with diabetes and hypertension, cardiac arrhythmias and hypertension, or other conditions may be automatically queried and stored on electronic storage 14. In some embodiments, user 36 may select a previous exploration effort and proceed from where the user left off.

Returning to FIG. 1, communications component 26 is configured to obtain a reference feedback related to the possible insight information (described below). In some embodiments, the reference feedback is obtained from a predefined database that indicates which attributes or properties have clinical relevance, given the context in which the data analysis is performed. In some embodiments, communications component 26 is configured to provide the reference feedback to the machine learning model to train the machine learning model. In some embodiments, communications component is configured to obtain user input related to the user 36's preference of the possible insight information. In some embodiments, communications component 26 is configured to provide the user input to the machine learning model to train the model on the user preferred insight information over time. In some embodiments, communications component 26 is configured to determine the preferences of user 36 by utilizing user 36's historical usage of the system and deriving from a frequency analysis which attributes are most interesting to him/her (more frequently queried attributes are more likely to be of interest than others). For example, communications component 26 may utilize a user voting system that obtains the preferences of user 36 and derives therefrom the (clinical) relevance of individual attributes or properties given a particular individual or a set of individuals. In this example, communications component 26 provides a voting option with each possible insight information generated (described below), stores all votes, and utilizes the votes as a weighing mechanisms for the possible insight information generated.

In some embodiments, communication component 26 is configured to facilitate determination of (clinical) meaningfulness of insights by implementing an understanding of the context via a rule based system (e.g., financial analysts focus on costs as the KPI (key performance indicator: e.g., one or more attributes) and are more interested in diagnostic information rather than vital signs).

In some embodiments, communications component 26 is configured to obtain information related to one or more attributes of a new individual. In some embodiments, the one or more attributes of the new individual includes one or more physiological parameters (e.g., vital signs), demographic information, or other information.

Clustering component 28 is configured to determine a grouping of a data collection (e.g., representative of a population of individuals) to obtain groups representative of a plurality of individuals. In some embodiments, each of the plurality of individuals have at least one attribute of a plurality of attributes. In some embodiments, clustering component 28 is configured to determine the grouping based on one or more thresholds of one or more variables (e.g., age-groups), random assignment, human preference, one or more clustering algorithms, or other information. In some embodiments, clustering component 28 is configured to perform clustering on the data collection. In some embodiments, clustering component 28 is configured to perform the clustering via a machine learning model (e.g., as described above). As an example, clustering component 28 may provide the data collection (or a portion thereof) as input to the machine learning model to cause the machine learning model to output the group information (e.g., identification of the groups, characteristics of the groups, characteristics of the individuals assigned to the groups, or other information related the groups). In some embodiments, the machine learning model is configured to determine which aspects of the data collection are important. In the context of clustering, the machine learning model determines when to consider two individuals similar or different from each other. In some embodiments, such determinations are made using one or more of hierarchical methods, centroid-based methods, prototype-based methods, distribution/density based methods, fuzzy variants method, metric learning methods, or other methods. In one embodiment, the present disclosure comprises means for performing clustering on the data collection to obtain groups representative of a plurality of individuals, with such means for performing clustering taking the form of clustering component 28.

In some embodiments, hierarchical methods facilitate the determinations by continuously looking for the smallest distances observed and then merging an individual with an already formed cluster. In some embodiments, centroid based methods facilitate the determinations by choosing groups such that their centroids (means/medians/modes) optimize some criterion. In some embodiments, prototype based methods facilitate the determinations by optimizing positions of some representatives of the population such that a criterion is optimized. In some embodiments, distribution/density based methods explicitly model the density of population data collection and identify areas where many individuals are densely together. In some embodiments, fuzzy variants facilitate the determinations based on a generalization that individuals may be member of multiple groups (represented by probabilities). In some embodiments, metric learning methods facilitate the determinations by optimizing the distance measures (used to define ‘close’/‘far’) with respect to the population data collection. By way of a non-limiting example, FIG. 3 illustrates hierarchical clustering of a population, in accordance with one or more embodiments. In FIG. 3, all individual patients are located at the bottom, and horizontal lines indicate which patients are joined to form groups (e.g., clusters).

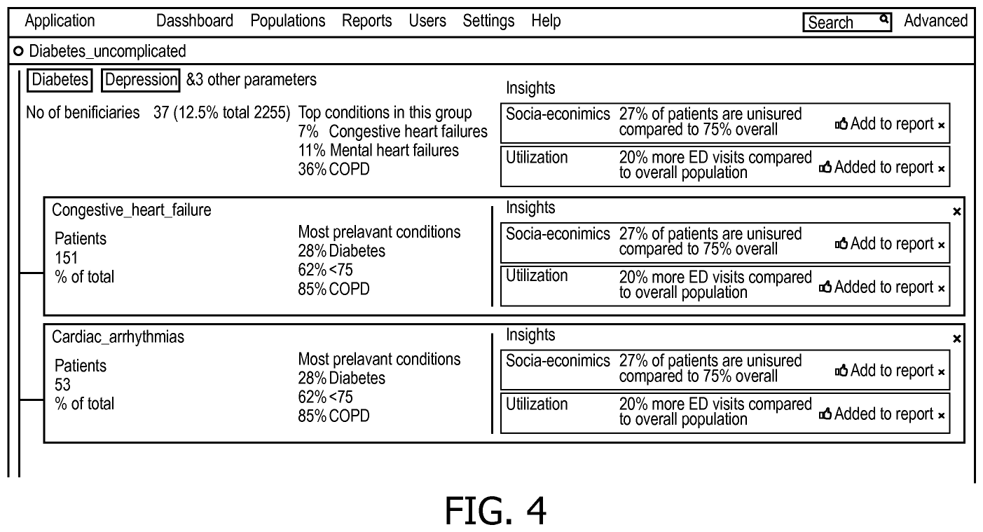

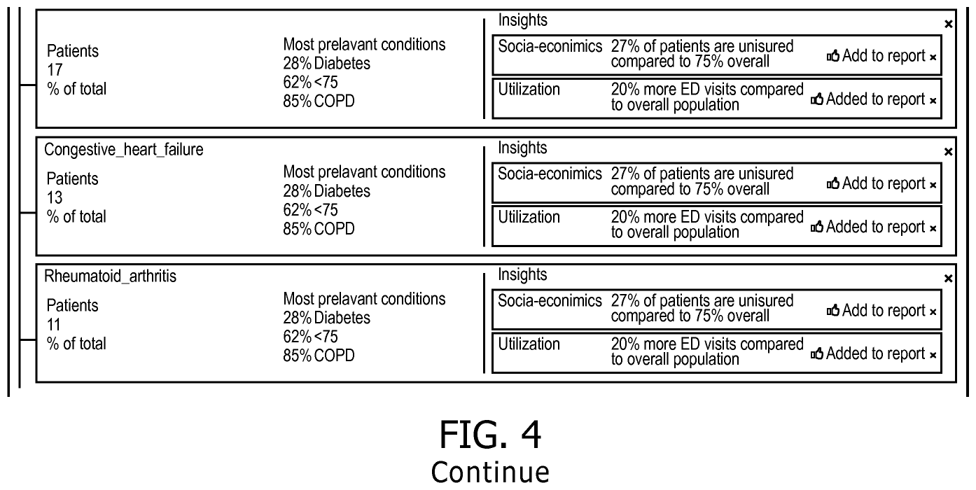

FIG. 4 illustrates different groups of patients having similar clinical conditions, in accordance with one or more embodiments. As shown in FIG. 4, the main group (Diabetes Uncomplicated) is segmented into several smaller subgroups of clinically similar patients based on their clinical conditions (i.e. congestive heart failure, cardiac arrhythmias, etc.). In FIG. 4, linked to both the overarching group and the smaller subgroups are insights that are based on available data on the main and the subgroups.

Returning to FIG. 1, in some embodiments, clustering component 28 is configured to determine which group the new individual identifies with based on the obtained information of the new individual.

In some embodiments, statistics component 30 is configured to determine a statistic for each attribute of the plurality of attributes for each of the groups. For example, statistics component 30 may determine mean and standard deviation for each attribute of each group. In some embodiments, depending on the type of data (continuous, ordinal, categorical, binary) and distribution (normal, lognormal, uniform, exponential), statistics component 30 may determine different statistics. In one embodiment, the present disclosure comprises means for determining a statistic, with such means for determining the statistic taking the form of statistics component 30.

In some embodiments, statistics component 30 is configured to determine, for each attribute of the plurality of attributes, whether there is a difference between the statistic of an attribute of a group and the statistics of the attribute of the other groups. In one embodiment, the present disclosure comprises means for determining whether there is a difference between the statistic of an attribute of a group and the statistics of the attribute of the other groups, with such means for determining the difference taking the form of statistics component 30. In some embodiments, statistics component 30 is configured to determine whether a measure of significance for each difference of the differences exceeds a predetermined threshold. For example, statistics component 30 applies a statistical test to determine the likelihood (p-value) that the statistics obtained from a pair of groups come from different underlying distributions. In this example, for each attribute and each combination of groups, statistics component 30 is configured to apply, based on the nature of the attribute, one or more statistical tests to determine a measure of significance (p-value) of the statistics. As an example, in case of continuous or categorical data, statistics component 30 applies the Wilcoxon rank-sum test. As another example, in case of binary data, statistics component 30 applies relative risk. In one embodiment, the present disclosure comprises means for determining whether a measure of significance for each difference of the differences exceeds a predetermined threshold, with such means for determining the measure of significance taking the form of statistics component 30.

In some embodiments, statistics component 30 is configured such that the relation or relations (e.g., between groups and insight information) lead to the formation of an action plan or an action. As such, statistics component 30 is configured to provide a database having attributes that are modifiable. In some embodiments, the modifiability of an attribute is determined by determining, over the lifetime of individuals, whether the attribute shows changes before and after interventions. For example, statistics component 30 is configured to identify interventions by selecting procedures and the dates they were performed from the data collection. In some embodiments, statistics component 30 is configured to determine the value of an attribute measured in a chosen time period before the procedure (e.g., a window of 1 week) and store the value as ‘pre’ value. In some embodiments, statistics component 30 is configured to determine the value for the attribute measured during a similar time period after the intervention and store the value as ‘post’ value. In some embodiments, statistics component is configured to determine for each pair of pre/post measurements the difference (post-pre) and perform a statistical test (e.g., t-test) to determine whether these differences are statistically significantly different from zero (e.g., a p-value of less than 0.2, a p-value of less than 0.05, etc.). In some embodiments, responsive to the differences being statistically significantly different from zero, statistics component 30 is configured to identify the attribute as modifiable.

In some embodiments, statistics component 30 is configured to determine a validity time interval for the insight information based on a longitudinal analysis of historical values corresponding to the attribute of one or more individuals. In some embodiments, statistics component 30 is configured to determine how volatile the insight information is by assessing how long the attribute keeps a stable value before a change occurs. In some embodiments, responsive to the validity time interval being short (e.g., less than 2 hours, less than 15 minutes, less than 1 minute, etc.), the insights information may lose its validity more quickly than when it is based on less volatile (more stable) attributes. In some embodiments, statistics component 30 is configured to perform a check that the values of more volatile attributes have been recently updated. In some embodiments, responsive to the volatile attribute values being outdated, statistics component 30 is configured to request for a data update before the insight information is generated.

In some embodiments, statistics component 30 is configured to determine reproducible differences. In some embodiments, statistics component 30 is configured to determine attribute differences multiple times on random sub-selections of the population and select the differences that are observed in the majority of calculations. For example, differences appearing in the top 25 of multiple calculations may be selected.

In some embodiments, insight generation component 32 is configured to generate insight information based on information indicating the differences that have significance levels exceeding the predetermined threshold. In some embodiments, insight generation component 32 may generate insight information via one or more prediction models (e.g., a neural network or other machine learning model). In some embodiments, insight generation component 32 may provide, for each difference of the differences that has the measure of significance exceeding the predetermined threshold, the difference as input to a machine learning model (or other prediction model) that has been previously trained on user 36's preferences regarding the possible insight information, clinical relevance of the insight information, or other information to cause the machine learning model to generate insight information. In some embodiments, the insight information reflects the difference between a type of individual and other types of individuals relative to the attribute. In some embodiments, the type of individual is associated with the group, and the other types of individuals are associated with at least one other group. In some embodiments, the type of individual has a set of characteristics, and the other types of individuals respectively have other sets of characteristics. In some embodiments, the insight information indicates a direction and a magnitude of the difference observed between individuals in one group and individuals in at least one other group. For example, differences having a p-value of greater than 0.20 (or other predetermined p-value threshold) may be provided to the machine learning model to generate insight information.

For example, the insight information may include “The cluster with mainly hypertension patients has 14% less cardiac arrhythmias than other patients,” “The cluster with mainly solid tumor and hypertension patients has 27% more solid tumor than other patients,” “The cluster with mainly hypertension and cardiac arrhythmias and chronic pulmonary patients has 45% more psychoses than other patients,” “The cluster with mainly hypertension patients has 16% less cardiac arrhythmias than the cluster with mainly diabetes uncomplicated and hypertension patients,” “The cluster with mainly hypothyroidism and hypertension patients has 27% less solid tumor than the cluster with mainly solid tumor and hypertension patients,” “The cluster with mainly rheumatoid arthritis and hypertension patients has 25% more valvular disease than the cluster with mainly hypertension and diabetes complicated patients,” or other insight information.

In some embodiments, information related to one or more attributes (e.g., physiological parameters, demographic parameters, etc.) of a new individual is obtained by communications component 26. In some embodiments, the new individual is classified, via clustering component 28, in one of the groups (e.g., as previously created) based on the obtained information and the groups characteristics. In some embodiments, insight generation component 32 is configured to generate insight information applicable to the new individual based on the new individual's identification with the determined group. For example, if the new individual identifies with group 1, insights comparing group 1 and other groups may be generated.

In some embodiments, insight generation component 32 is configured to initiate, based on the insight information applicable to the new individual, a predetermined intervention. For example, the insight information applicable to the new individual may include 60% higher hospitalization associated with elevated heart rates. Accordingly, insight generation component 32 may initiate a medical intervention (e.g., determine a breathing regimen, prescribe a medication, propose a diet change) to mitigate further hospitalization.

In some embodiments, insight generation component 32 may generate possible insight information via one or more prediction models (e.g., a neural network or other machine learning model). In some embodiments, insight generation component 32 may provide, for each difference of the differences that has the measure of significance exceeding the predetermined threshold, the difference as input to a machine learning model (or other prediction model) that has not been previously trained on user 36's preferences regarding the possible insight information, clinical relevance of the insight information, or other information to cause the machine learning model to generate possible insight information.

The possible insight information includes any novel, interesting, plausible, and understandable relation, or set of associated relations, that is selected from a larger set of relations derived from the data collection. Based on the nature of some the possible insight information, a user (e.g., clinician, healthcare providers) may not be able to act on such possible insight information. For example a possible insight information describing a higher likelihood of a person with blue eyes spending more money for X-Rays may not be actionable as a person's eye color cannot be changed. As another example, a possible insight information describing a higher likelihood of a person with high blood pressure spending more for hospital stays is actionable as a user (e.g., clinician, care provider) may take steps (e.g., medical interventions) to minimize hospitalizations. As such, insight generation component 32 is configured to train the machine learning model (e.g., based on clinical data base, based on historical user preferences) to generate actionable insight information. For example, insight generation component 32 is configured to differentiate, via the machine learning model, the insight information from the possible insight information.

In one embodiment, the present disclosure comprises means for generating insight information, with such means for generating insight information taking the form of insight generation component 32.

Presentation component 34 is configured to effectuate, via user interface 20, presentation of the insight information. In some embodiments, presentation component 34 is configured to effectuate, via user interface 20, presentation of the possible insight information to user 36. In some embodiments, presentation component 34 is configured to generate the insight information in a human readable format (e.g., natural language generation). In some embodiments, the human readable format includes a textual representation (i.e., a well formed and grammatically correct English sentence) that describes the difference relative to the attribute. For example, presentation component 34 may use Pattern 1 to generate the insight information.

Pattern 1: “The cluster with mainly <cl1_description> patients has <pct>% <more/less> <characteristic_name> than the cluster with mainly <cl2_description> patients.”

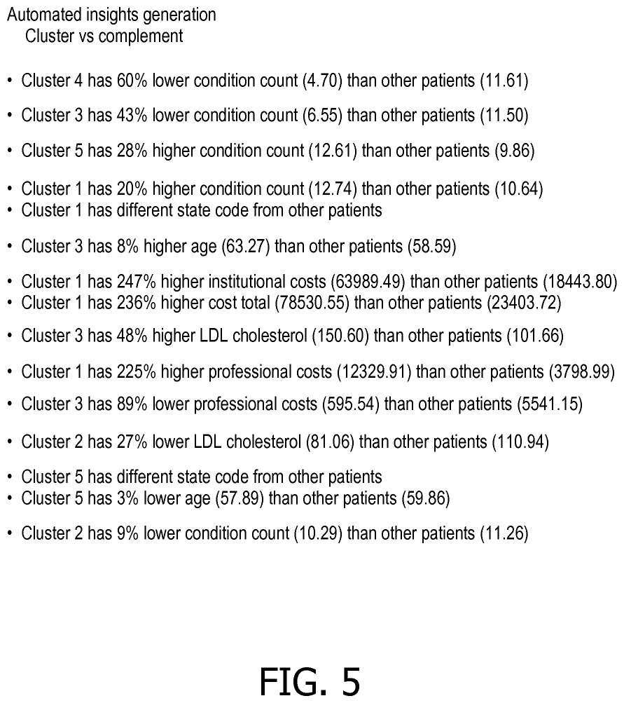

By way of a non-limiting example, FIG. 5 illustrates insight information generated for a cluster vs. a complement, in accordance with one or more embodiments. As shown in FIG. 5, a single cluster is compared with the other clusters (e.g., combined). In this example, the insight information generated may include information related to an overall category of attributes (e.g., condition count, state code, age, cost, etc.).

In some embodiments, presentation component 34 is configured to generate insight information related to a cluster vs a complement (e.g., all of the other groups) which specifies particular attributes of comparison. For example, presentation component 34 may use Pattern 2 to generate such insight information.

Pattern 2: “Cluster <nr> has <pct>% more/less <characteristic name> (<value>) than other patients.”

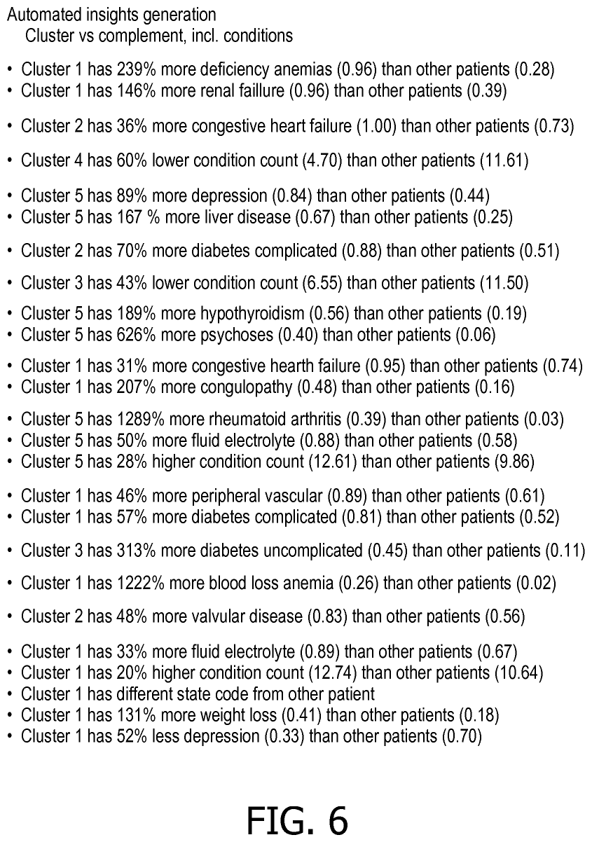

By way of a non-limiting example, FIG. 6 illustrates insight information generated for a cluster vs. a complement that specifies the particular attribute of comparison, in accordance with one or more embodiments. As shown in FIG. 6, a first cluster is compared to the other clusters (e.g., combined groups). The insight information related to such comparison is generated with the specific attribute of the comparison (e.g., deficiency anemia, congestive heart failure, etc.).

In some embodiments, presentation component 34 is configured to generate insight information related to a comparison of peer clusters. For example, a first cluster may be compared to a second cluster with respect to a particular attribute. In this example, presentation component 34 may use Pattern 3 to generate the insight information.

Pattern 3: “Cluster <nr> has <pct>% more/less <characteristic name> (<value>) than cluster <nr> (<value>).”

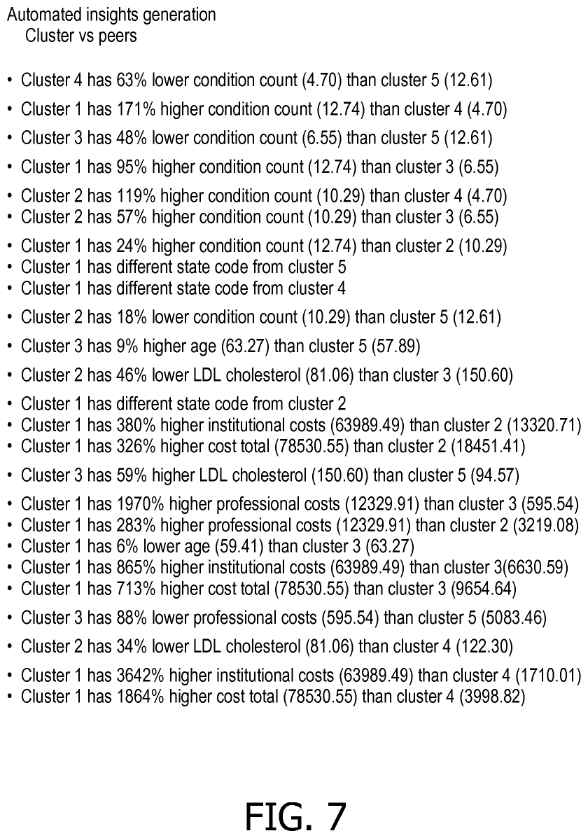

By way of a non-limiting example, FIG. 7 illustrates insight information generated for a cluster vs. other peer clusters, in accordance with one or more embodiments. As shown in FIG. 7, a single cluster is compared with the other individual clusters. In this example, the insight information generated may include information related to an overall category of attributes (e.g., condition count, state code, age, cost, etc.) or include specific attributes of comparison.

In some embodiments, presentation component 34 is configured to combine multiple insights describing a single group and create natural language generation patterns that can formulate a combined insight. For example, insight information having multiple insights combine may be presented as “Cluster 1 shows high costs, which might be linked to the patients having deficiencies/anemia and/or renal failure; they also primarily live in Florida.”

In one embodiment, the present disclosure comprises means for effectuating presentation of the insight information, with such means for effectuating presentation of the insight information taking the form of presentation component 34.

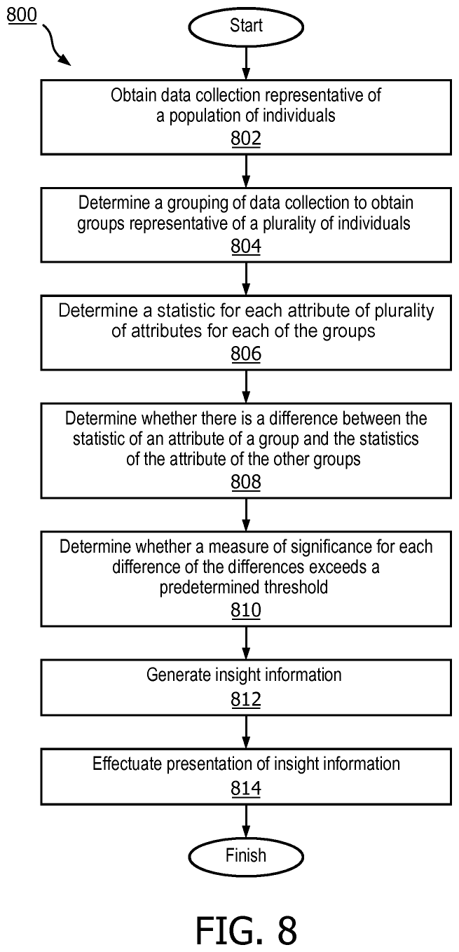

FIG. 8 illustrates a method 800 for providing model-based population insight generation, in accordance with one or more embodiments. Method 800 may be performed with a system. The system comprises one or more processors, or other components. The processors are configured by machine readable instructions to execute computer program components. The computer program components include a communications component, a clustering component, a statistics component, an insight generation component, a presentation component, or other components. The operations of method 800 presented below are intended to be illustrative. In some embodiments, method 800 may be accomplished with one or more additional operations not described, or without one or more of the operations discussed. Additionally, the order in which the operations of method 800 are illustrated in FIG. 8 and described below is not intended to be limiting.

In some embodiments, method 800 may be implemented in one or more processing devices (e.g., a digital processor, an analog processor, a digital circuit designed to process information, an analog circuit designed to process information, a state machine, or other mechanisms for electronically processing information). The devices may include one or more devices executing some or all of the operations of method 800 in response to instructions stored electronically on an electronic storage medium. The processing devices may include one or more devices configured through hardware, firmware, or software to be specifically designed for execution of one or more of the operations of method 800.

At an operation 802, a data collection representative of a population of individuals is obtained. In some embodiments, operation 802 is performed by a processor component the same as or similar to communications component 26 (shown in FIG. 1 and described herein).

At an operation 804, a grouping of the data collection is determined to obtain groups representative of a plurality of individuals. In some embodiments, each of the plurality of individuals have at least one attribute of a plurality of attributes. In some embodiments, operation 804 is performed by a processor component the same as or similar to clustering component 28 (shown in FIG. 1 and described herein).

At an operation 806, a statistic is determined for each attribute of the plurality of attributes for each of the groups. In some embodiments, operation 806 is performed by a processor component the same as or similar to statistics component 30 (shown in FIG. 1 and described herein).

At an operation 808, for each attribute of the plurality of attributes, it is determined whether there is a difference between the statistic of an attribute of a group and the statistics of the attribute of the other groups. In some embodiments, operation 808 is performed by a processor component the same as or similar to statistics component 30 (shown in FIG. 1 and described herein).

At an operation 810, it is determined whether a measure of significance for each difference of the differences exceeds a predetermined threshold. In some embodiments, operation 810 is performed by a processor component the same as or similar to statistics component 30 (shown in FIG. 1 and described herein).

At an operation 812, for each difference of the differences that has the measure of significance exceeding the predetermined threshold, insight information is generated. In some embodiments, the insight information reflects the difference between a type of individual and other types of individuals relative to the attribute. In some embodiments, the type of individual is associated with the group, and the other types of individuals are associated with at least one other group. In some embodiments, operation 812 is performed by a processor component the same as or similar to insight generation component 32 (shown in FIG. 1 and described herein).

At an operation 814, the insight information is presented via a user interface. In some embodiments, operation 814 is performed by a processor component the same as or similar to presentation component 34 (shown in FIG. 1 and described herein).

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

1. A system for managing a medical resource, the system comprising:
one or more processors configured by machine-readable instructions to:
obtain a data collection representative of a population of individuals;
determine a grouping of the data collection to obtain groups representative of a plurality of individuals, each of the plurality of individuals having at least one attribute of a plurality of attributes;
determine a statistic for each attribute of the plurality of attributes for each of the groups;
for each attribute of the plurality of attributes, determine whether there is a difference between the statistic of an attribute of a group and the statistics of the attribute of the other groups;
determine whether a measure of significance for each difference of the differences exceeds a predetermined threshold;
generate, for each difference of the differences that has the measure of significance exceeding the predetermined threshold, insight information, the insight information reflecting the difference between a type of individual and other types of individuals relative to the attribute, the type of individual being associated with the group, and the other types of individuals being associated with at least one other group; and
manage the medical resource as a function of the insight information.

2. The system of claim 1, wherein the one or more processors are further configured to:
obtain information related to one or more attributes of a new individual;
determine which group the new individual identifies with based on the obtained information of the new individual; and
generate insight information applicable to the new individual based on the new individual's identification with the determined group.

3. The system of claim 2, wherein the one or more processors are further configured to initiate a predetermined intervention based on the insight information applicable to the new individual.

4. The system of claim 1, wherein the one or more processors are further configured to:
for each difference of the differences that has the measure of significance exceeding the predetermined threshold, provide the difference as input to a machine learning model to cause the machine learning model to generate possible insight information;
obtain a reference feedback related to the possible insight information;
provide the reference feedback to the machine learning model to train the machine learning model; and
generate, via the machine learning model, the insight information.

5. The system of claim 4, wherein the one or more processors are configured to:
effectuate presentation of the possible insight information to a user;
obtain user input related to the user's preference of the possible insight information;
provide the user input to the machine learning model to train the model on the user preferred insight information over time; and
differentiate, via the machine learning model, the insight information from the possible insight information.

6. The system of claim 1, wherein the one or more processors are further configured to determine a validity time interval for the insight information based on a longitudinal analysis of historical values corresponding to the attribute of one or more individuals.

7. A method for managing a medical resource, the method comprising:
obtaining, with one or more processors, a data collection representative of a population of individuals;
determining, with the one or more processors, a grouping of the data collection to obtain groups representative of a plurality of individuals, each of the plurality of individuals having at least one attribute of a plurality of attributes;
determining, with the one or more processors, a statistic for each attribute of the plurality of attributes for each of the groups;
for each attribute of the plurality of attributes, determining, with the one or more processors, whether there is a difference between the statistic of an attribute of a group and the statistics of the attribute of the other groups;
determining, with the one or more processors, whether a measure of significance for each difference of the differences exceeds a predetermined threshold;
for each difference of the differences that has the measure of significance exceeding the predetermined threshold, generating, with the one or more processors, insight information, the insight information reflecting the difference between a type of individual and other types of individuals relative to the attribute, the type of individual being associated with the group, and the other types of individuals being associated with at least one other group; and
manage the medical resource as a function of the insight information.

8. The method of claim 7, further comprising:
obtaining, with the one or more processors, information related to one or more attributes of a new individual;
determining, with the one or more processors, which group the new individual identifies with based on the obtained information of the new individual; and
generating insight information applicable to the new individual based on the new individual's identification with the determined group.

9. The method of claim 8, further comprising initiating, with the one or more processors, a predetermined intervention based on the insight information applicable to the new individual.

10. The method of claim 7, further comprising:
for each difference of the differences that has the measure of significance exceeding the predetermined threshold, providing, with the one or more processors, the difference as input to a machine learning model to cause the machine learning model to generate possible insight information;
obtaining, with the one or more processors, a reference feedback related to the possible insight information;
providing, with the one or more processors, the reference feedback to the machine learning model to train the machine learning model; and
generating, via the machine learning model, the insight information.

11. The method of claim 10, further comprising:
effectuating, via the user interface, presentation of the possible insight information to a user;
obtaining, with the one or more processors, user input related to the user's preference of the possible insight information;
providing, with the one or more processors, the user input to the machine learning model to train the model on the user preferred insight information over time; and
differentiating, via the machine learning model, the insight information from the possible insight information.

12. The method of claim 7, further comprising determining, with the one or more processors, a validity time interval for the insight information based on a longitudinal analysis of historical values corresponding to the attribute of one or more individuals.

13. A system for providing model-based population insight generation, the system comprising:
means for obtaining a data collection representative of a population of individuals;
means for determining a grouping of the data collection to obtain groups representative of a plurality of individuals, each of the plurality of individuals having at least one attribute of a plurality of attributes;
means for determining a statistic for each attribute of the plurality of attributes for each of the groups;
for each attribute of the plurality of attributes, means for determining whether there is a difference between the statistic of an attribute of a group and the statistics of the attribute of the other groups;
means for determining whether a measure of significance for each difference of the differences exceeds a predetermined threshold;
for each difference of the differences that has the measure of significance exceeding the predetermined threshold, means for generating insight information, the insight information reflecting the difference between a type of individual and other types of individuals relative to the attribute, the type of individual being associated with the group, and the other types of individuals being associated with at least one other group; and
means for effectuating presentation of the insight information.

14. The system of claim 13, further comprising:
means for obtaining information related to one or more attributes of a new individual;
means for determining which group the new individual identifies with based on the obtained information of the new individual; and
means for generating insight information applicable to the new individual based on the new individual's identification with the determined group.

15. The system of claim 14, further comprising means for initiating a predetermined intervention based on the insight information applicable to the new individual.

16. The system of claim 13, further comprising:
for each difference of the differences that has the measure of significance exceeding the predetermined threshold, means for providing the difference as input to a machine learning model to cause the machine learning model to generate possible insight information;
means for obtaining a reference feedback related to the possible insight information;
means for providing the reference feedback to the machine learning model to train the machine learning model; and
means for generating the insight information.

17. The system of claim 16, further comprising:
means for effectuating presentation of the possible insight information to a user;
means for obtaining user input related to the user's preference of the possible insight information;
means for providing the user input to the machine learning model to train the model on the user preferred insight information over time; and
means for differentiating the insight information from the possible insight information.

18. The system of claim 13, further comprising means for determining a validity time interval for the insight information based on a longitudinal analysis of historical values corresponding to the attribute of one or more individuals.
